(19) United States
(12) Patent Application Publication
(10) Pub. No.: US 2025/0201390 A1
(43) Pub. Date: Jun. 19, 2025

(54) METHODS OF INCORPORATING SPORADIC OBJECTS ONTO AN EXISTING MESH

(71) Applicant: YALE UNIVERSITY, New Haven, CT (US)

(72) Inventor: Daniel Pak, New Haven, CT (US)

(21) Appl. No.: 18/987,814

(22) Filed: Dec. 19, 2024

Related U.S. Application Data
(60) Provisional application No. 63/611,903, filed on Dec. 19, 2023.

(51) Int. Cl. G16H 30/40; G06T 17/20; G06T 19/20

(52) U.S. Cl. CPC G16H 30/40; G06T 17/205; G06T 19/20; G06T 2219/2021

(57) ABSTRACT

Provided herein are automated methods of incorporating sporadic objects onto an existing object mesh with anatomical consistency. The method includes providing an existing object (EO) mesh; providing a voxelgrid segmentation of sporadic objects (SO); applying an SO meshing algorithm, the SO meshing algorithm generating an SO surface mesh from the EO mesh and the SO voxelgrid segmentation; wherein the SO mesh has complete mesh correspondence with the EO mesh along contact surfaces; wherein the SO mesh is free of mesh degeneracy; and wherein the EO mesh topology is unchanged.

### CROSS-REFERENCE TO RELATED APPLICATIONS

The present application claims priority under 35 U.S.C. § 119(e) to U.S. Provisional Patent Application No. 63/611,903, filed Dec. 19, 2023, which application is incorporated herein by reference in its entirety.

### STATEMENT REGARDING FEDERALLY SPONSORED RESEARCH OR DEVELOPMENT

This invention was made with government support under F31HL162505 awarded by National Institutes of Health (NIH). The government has certain rights in the invention.

### BACKGROUND

In medical imaging, meshing is a crucial step for various 3D modeling tasks, such as shape analysis, physics-driven simulations, graphics, and 3D printing. For sporadic objects with vastly different inter-patient shape characteristics (e.g. volume, curvature, connectivity, etc.), the most common meshing approach is to first obtain their voxelgrid segmentation and then to convert the segmentation into a surface mesh using isosurface extraction techniques. However, the naïve application of this approach often neglects the surrounding environment where meshes of different objects may already exist. Consequently, the final set of meshes must often be post-processed using various expert-driven editing tools before it can be used for the desired tasks. The need for the manual mesh editing steps severely limits the speed and accuracy of any modeling tasks, as the mesh editing processes are often time-consuming and difficult to master.

The problem is further exacerbated when the user wishes to preserve the topology of the existing mesh. Such a scenario is commonly presented when mesh registration is performed to preserve the original mesh topology across intra- and inter-patient spatiotemporal domains. This is relevant for tasks including, but not limited to, longitudinal studies, population-based studies, and motion tracking. After registration has been performed, preserving the original mesh topology is not only convenient, but often necessary to perform the desired tasks. Many existing tools are not fit for this task because they often alter the original mesh topology to obtain the desired mesh qualities.

Accordingly, there is a need in the art for articles and methods that improve on existing articles and methods for incorporating sporadic objects onto an existing mesh. The present invention addresses this need.

### SUMMARY

In one aspect, an automated method of incorporating sporadic objects (SO) onto an existing object (EO) mesh, the method including providing an EO mesh; providing an SO voxelgrid segmentation; and applying an SO meshing algorithm, the SO meshing algorithm generating an SO surface mesh from the combination of the EO mesh and the SO voxelgrid segmentation; wherein the SO mesh has complete mesh correspondence with the EO mesh along contact surfaces, the SO mesh is free of mesh degeneracy, and the EO mesh topology is unchanged. In some embodiments, the EO mesh includes a surface mesh, a volumetric mesh, or a combination thereof.

In some embodiments, the method further includes post-processing the SO voxelgrid segmentation, the post-processing removing undesired spatial gaps between the SO voxelgrid segmentation and the EO mesh. In some embodiments, the post-processing step includes converting the EO mesh into EO voxelgrid segmentation; dilating the SO voxelgrid segmentation; keeping only segments of the EO voxelgrid segmentation that intersect with segments of the dilated SO voxelgrid segmentation; combining the EO and SO voxelgrid segmentations; morphologically closing the combined voxelgrid segmentations; and filtering the combined voxelgrid segmentations by subtracting the EO segments and performing volume-based island removals. In some embodiments, the SO voxelgrid segmentation includes calcification segments. In some embodiments, the method further includes, prior to the converting step, splitting the calcification segments into disjoint groups to prevent over-dilation (i.e. overly connected components). In some embodiments, the dilation and morphological closing operations are performed using combinations of isotropic and/or anisotropic filters. In some embodiments, the anisotropy is defined as the major principal axis pointing in the same direction as the nearest EO mesh node's surface normal.

In some embodiments, the SO meshing algorithm includes generating a background tetrahedral mesh; converting the SO segmentation to a nodal signed distance function (SDF); generating an isosurface triangular mesh from the background mesh and nodal SDF using marching tetrahedra; and simultaneously optimizing the nodal SDF and the background mesh, forming an optimized SO surface mesh.

In some embodiments, generating the background mesh includes extracting the EO surface; processing the surface for constrained tetrahedral meshing; performing constrained tetrahedral meshing; and adding a fake node to all background boundary surface elements to form fake tetrahedral elements. In some embodiments, generating the background mesh further includes after extracting the EO surface, generating an offset surface including all areas within a pre-defined voxel spacing away from the surface of the region of interest; combining the extracted surface and the offset surface, forming merged surfaces for tetrahedral meshing; performing the constrained tetrahedral meshing with the merged surfaces; and before adding the fake node, removing elements inside the original EO surface.

In some embodiments, converting the SO segmentation to a nodal signed distance functions (SDF) includes converting the SO segmentation to a voxelgrid SDF via linear mapping or voxelwise distance calculation from the SO isosurface; interpolating the voxelgrid SDF at each background mesh node, forming nodal SDF.

In some embodiments, optimizing the nodal SDF and the background mesh includes adjusting the nodal SDF and the mesh vertex positions to maximize surface mesh quality and minimize deviation from the SO voxelgrid segmentation. In some embodiments, the method further includes setting the nodal SDF values to prescribed values when the nodes are from the original EO mesh nodes or the fake node.

In some embodiments, the method further includes remeshing non-contact surfaces after generating the isosurface triangular mesh of the SO surface, the remeshing of the non-contact surfaces reducing complexity of the mesh. In some embodiments, the remeshing includes constrained surface remeshing. In some embodiments, the constrained surface remeshing includes splitting the optimized SO surface mesh into two disjoint sets of contact and non-contact elements, each set of elements representing a separate surface mesh; determining that an element is a contact element when all three nodes of a triangular element are coincident with the original mesh nodes; performing an initial vertex-based clustering step on the non-contact surface mesh; reassigning the clusters at overlapping nodes between the contact and non-contact surface meshes; performing a standard vertex clustering-based triangulation, providing a remeshed non-contact surface mesh; and merging the contact surface mesh and the remeshed non-contact surface mesh to obtain a final output surface mesh. In some embodiments, the method further includes repeating the remeshing to achieve a desired element size and density.

In some embodiments, the method further includes, after applying the SO meshing algorithm, applying a tetrahedralization algorithm to the SO surface mesh, the tetrahedralization algorithm converting the SO surface mesh to a tetrahedral SO volumetric mesh; and replacing the SO surface mesh with the SO volumetric mesh in the subsequent merging step.

In some embodiments, the method further includes merging the SO surface/volumetric mesh with the EO mesh via coincident nodes.

In another aspect, an apparatus for automated calcification meshing with anatomical consistency includes a processor; a memory unit; and a communication interface; wherein the processor is connected to the memory unit and the communication interface and the processor and memory are configured to implement the method according to any of the embodiments disclosed herein.

In a further aspect, provided herein is a non-transitory computer readable storage medium storing computer-executable instructions for performing the method according to any of the embodiments disclosed herein.

### BRIEF DESCRIPTION OF THE DRAWINGS

FIG. 1 shows images illustrating that C-MAC robustly and automatically incorporates a voxelgrid segmentation into an existing mesh without changing the original mesh topology. Here, we demonstrate its performance using two vastly different calcification segmentations (orange) and complex aortic valve meshes (gray).

FIG. 2 shows a schematic illustrating the main overview of C-MAC. Starting from a 3D CTA, a patient-specific heart volumetric mesh is first generated using DeepCarve. The resulting heart mesh is used to aid the voxelgrid segmentation of calcification, as well as to generate the background mesh for DMTet. The SDF values are first initialized by sampling the voxelgrid segmentation at each node of the background grid, and subsequently the node coordinates and nodal SDF values are optimized for better element quality. The resulting DMTet surface mesh is further processed via node-based remeshing to generate the final input for tetrahedralization. The details of each process can be found in the main text and later figures.

FIG. 3 shows images highlighting the effects of PostProcessCa2Seg on one test-set patient. Yellow: calcification, blue: aortic wall, (green, orange, purple): aortic valve leaflets. The first two columns are two different 2D-slice views, and the third column is a 3D view of the aortic valve.

FIG. 4 shows images illustrating the background mesh generation process. From left to right: (a) patient-specific mesh of the aorta+aortic valve leaflets, (b) TetGen input generated by combining the exterior surface and an offset surface from the original geometry, (c) preliminary background mesh generated by TetGen and hollowing, (d) final background mesh after adding a “fake” vertex to the exterior surface elements of the preliminary mesh. All meshes are clipped at a viewing plane for visualization purposes.

FIG. 5 shows images illustrating the three main sequential steps for anatomically consistent surface meshing. From left to right: (a) initial inputs of patient-specific aorta+aortic valve leaflets (gray) and voxelgrid segmentation of calcification (red), (b) initial DMTet mesh with raw sampled SDF, (c) optimized DMTet mesh, (d) final remeshed surface. Red box indicates the viewing region, and colored circles indicate noticeable regions of improvement after each step.

FIGS. 6A-C shows images illustrating qualitative evaluation of the spatial accuracy of calcification using two image slice views for 3 test-set patients. GT: ground-truth segmentation, DL: deep learning segmentation using “GDL (ours)”, post: result of PostProcessCa2Seg using either GT or DL as the initial segmentation, final: result of the full C-MAC. Yellow: calcification, red: partial LV myocardium, blue: aorta, (green, orange, purple): aortic valve leaflets.

FIG. 7 shows images illustrating baseline comparisons for the final mesh quality. Yellow: calcification, green: aortic valve leaflet. Top: front view, bottom: back view. Colored circles indicate the noticeable regions of improvement from each baseline to C-MAC.

FIG. 8 shows a schematic illustrating the effect of PostProcessCa2Seg on the final C-MAC mesh. Yellow: calcification, green: aortic valve leaflet, purple dots: merged nodes. This example illustrates both the benefit and drawback of our postprocessing algorithm. Benefit: improved anatomical consistency with the surrounding heart tissue. Drawback: some regions with overestimated calcification.

FIG. 9 shows images illustrating valve opening simulations that demonstrate the effects of calcification on the final leaflet positions. Yellow is the ground-truth calcification, left is the input valve geometry predicted by DeepCarve, and right is the deformed geometry after FEA. Movement is clearly restricted near calcified regions.

FIG. 10 shows images illustrating stress (top) and strain (bottom) analyses from valve opening simulations. Left is the Gaussian KDE plot of stress/strain vs. distance to calcification from the aggregate of 35 test-set patient simulations. Right is one test-set patient with stress/strain overlaid with the valve leaflets, plus the ground-truth calcification (gray) for reference.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “mesh,” as used herein, refers to a collection of mesh elements, each of which is defined by a set of nodes and edges.

The term “surface mesh,” as used herein, refers to a mesh comprised entirely of two-dimensional (2D) elements. The space enclosed by the nodes and edges of 2D mesh elements has surface area, but no volume (e.g., a triangular element with 3 nodes and 3 edges).

The term “volumetric mesh,” as used herein, refers to a mesh comprised of three-dimensional (3D) elements. The space enclosed by the nodes and edges of 3D mesh elements has volume (e.g., a tetrahedral element with 4 nodes and 6 edges).

The term “voxelgrid segmentation,” as used herein, refers to elements using occupancy values within rectilinear 3D grids (a.k.a. voxelgrids) instead of nodes and edges to represent 3D geometry.

### DETAILED DESCRIPTION

Provided herein are methods for automatically incorporating sporadic objects (SO) onto the mesh of an existing object (EO). The EO includes any suitable object for modelling in mesh form, such as, but not limited to, tissues and/or organs. For example, in one embodiment, the EO includes the heart and/or related structures. The SO includes any suitable object formed on, in, or in connection with the EO. For example, in one embodiment, the SO includes plaque and/or calcification. In another embodiment, the EO includes the aortic wall and the SO includes dissection flaps. As will be appreciated by those skilled in the art, the EO and SO are not limited to the examples above and may include any other suitable object for modelling in mesh form and any other suitable object formed on, in, or in connection with the EO, respectively.

In some embodiments, the method includes providing a surface and/or volumetric EO mesh; providing an SO voxelgrid segmentation; and applying an SO meshing algorithm, the SO meshing algorithm generating an SO surface mesh from the combination of the EO mesh and the SO voxelgrid segmentation. In some embodiments, after applying the SO meshing algorithm, the method includes merging the SO surface mesh with the EO mesh via coincident nodes. Additionally or alternatively, in some embodiments, after applying the SO meshing algorithm, the method includes applying a tetrahedralization algorithm to the SO surface mesh. The tetrahedralization algorithm converts the SO surface mesh to a tetrahedral SO volumetric mesh. In such embodiments, the SO volumetric mesh replaces the SO surface mesh in the merging step.

In some embodiments, the method includes post-processing the SO voxelgrid segmentation prior to applying the SO meshing algorithm. The post-processing step includes removing undesired spatial gaps between the voxelgrid segmentation and the EO mesh. In some embodiments, the post-processing step includes converting the EO mesh into EO voxelgrid segmentation; dilating the SO voxelgrid segmentation; keeping only the segments of the EO voxelgrid segmentation that intersect with the segments of the dilated SO voxelgrid segmentation; combining the EO and SO voxelgrid segmentations; morphologically closing the combined voxelgrid segmentations; and filtering the combined voxelgrid segmentations by subtracting the EO segments and performing volume based island removals. In some embodiments, prior to the converting step, the method includes splitting the SO segments into disjoint groups to prevent over-dilation (i.e. overly connected components). In some embodiments, the closing includes a two-step morphological close. Additionally or alternatively, in some embodiments, the dilation and morphological closing operations are performed using different combinations of isotropic and/or anisotropic filters, where the anisotropy may be defined as the major principal axis pointing in the same direction as the nearest EO mesh node's surface normal.

In some embodiments, the SO meshing algorithm includes generating a background tetrahedral mesh; converting the SO segmentation to a nodal signed distance function (SDF); generating an isosurface triangular mesh from the background mesh and nodal SDF using marching tetrahedra; and simultaneously optimizing the nodal SDF and background mesh, forming an optimized SO surface mesh. The SO segmentation is optionally post-processed before being converted to a nodal SDF (i.e., the SO segmentation may be converted with or without post-processing). In some embodiments, the SO mesh generated by the SO meshing algorithm has complete mesh correspondence with the EO mesh along contact surfaces, is free of mesh degeneracy, and/or the EO mesh topology is unchanged. In some embodiments, the simultaneous optimization of the nodal SDF and background mesh improves the mesh quality. For example, in some embodiments, optimizing the nodal SDF and the background mesh includes adjusting the nodal SDF and the mesh vertex positions to maximize surface mesh quality and minimize deviation from the SO voxelgrid segmentation. Additionally or alternatively, in some embodiments, the method includes setting the nodal SDF values to prescribed values when the nodes are from the original EO mesh nodes and/or the fake node. The prescribed values are set independently for the EO mesh node and the fake node, and may be the same or different. For example, in one embodiment, different prescribed values are set for the EO mesh node and the fake node. In another embodiment, prescribed values are only set for the fake node and not the EO mesh node, or vice versa.

In some embodiments, generating the background mesh includes extracting the EO surface; processing the surface for constrained tetrahedral meshing; performing constrained tetrahedral meshing; and adding a fake node to all background boundary surfaces to form fake tetrahedral elements. Additionally or alternatively, in some embodiments, generating the background mesh further includes, after extracting the EO surface, generating an offset surface including all areas within a pre-defined voxel spacing away from the surface of the region of interest; combining the extracted surface and the offset surface, forming merged surfaces for tetrahedral meshing; performing the constrained tetrahedral meshing with the merged surfaces; and, before adding the fake node, removing elements inside the original EO surface.

In some embodiments, converting the SO segmentation to a nodal signed distance functions (SDF) includes converting the SO segmentation to a voxelgrid SDF via linear mapping or voxelwise distance calculation from the SO isosurface; and interpolating the voxelgrid SDF at each background mesh node, forming nodal SDF.

In some embodiments, the method further includes remeshing the non-contact surfaces after generating the isosurface triangular mesh of the SO surface, the remeshing of the non-contact surfaces reducing complexity of the mesh. The remeshing includes any suitable remeshing, such as, but not limited to, constrained surface remeshing. For example, in some embodiments, the constrained surface remeshing includes splitting the optimized SO surface into two disjoint sets of contact and non-contact elements, each set of elements representing a separate surface mesh; determining that an element is a contact element when all three nodes of a triangular element are coincident with the original mesh nodes; performing an initial vertex-based clustering step on the non-contact surface mesh; reassigning the clusters at overlapping nodes between the contact and non-contact surface meshes. performing a standard vertex clustering-based triangulation, providing a remeshed non-contact surface mesh; and merging the contact surface mesh and the remeshed non-contact surface mesh to obtain a final output surface mesh. In some embodiments, the method further includes repeating the remeshing to achieve a desired element size and density.

Also provided herein is an apparatus for automated calcification meshing with anatomical consistency. In some embodiments, the apparatus includes a processor, a memory unit, and a communication interface. The processor is connected to the memory unit and the communication interface, and the processor and memory are configured to implement the method of any one of the previous claims.

Further provided herein is a non-transitory computer readable storage medium storing computer-executable instructions for performing the method according to one or more of the embodiments disclosed herein.

The articles and methods disclosed herein provide an automated algorithm that can incorporate a new mesh of sporadic objects into an existing mesh. In some embodiments, the method incorporates the SO without altering the EO mesh topology. In contrast to mesh boolean operations, which are notoriously unreliable, especially when combined with complex downstream tasks like mesh-based simulations, the methods disclosed herein are fully automated and the outputs are very robust. The final mesh has great mesh quality and great contact surface definitions. Additionally, the original mesh's nodes and edges are never altered, which enables large-scale downstream tasks. Without wishing to be bound by theory, it is believed that the methods disclosed herein facilitate automated meshing in fast-paced settings, such as hospitals. For example, in some embodiments, the articles and methods disclosed herein may be used for calcification modeling, which provides more accurate modelling of various diseases.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

Calcification has significant influence over cardiovascular disease progression and intervention. Particularly in aortic stenosis and Transcatheter Aortic Valve Replacements (TAVR), the high-stress regions of interest often correspond to regions with high calcification. Although plenty of studies have proposed segmentation algorithms for calcification, their use in downstream engineering tasks often requires extensive manual and/or error-prone mesh editing operations to incorporate them into a given heart geometry. In this work, we propose C-MAC (Calcification Meshing with Anatomical Consistency): a novel end-to-end framework for robust aortic calcification segmentation and meshing from computed tomography angiography (CTA). First, we present a deep learning model for the initial calcification segmentation and a post-processing algorithm that encourages anatomical consistency. Second, we propose a robust meshing algorithm that converts the segmentation into volume meshes with complete contact surface matching with a given heart geometry. We leveraged recent advances in deep learning-based heart geometry reconstruction, Deep Marching Tetrahedra, and constrained meshing via TetGen. The algorithm is fully automated, and the results can be further improved by editing the input segmentation as needed. Our final output is a patient-specific calcified heart mesh that can be directly used for large-batch solid mechanics simulations. Our code is available at https://github.com/danpak94/Deep-Cardiac-Volumetric-Mesh.

### 1. INTRODUCTION

Calcification plays an important role in the development and progression of cardiovascular diseases, such as aortic stenosis and atherosclerosis. Accurate modeling of calcification has thus been a long-standing clinical interest (Sangiorgi et al., 1998; Pawade et al., 2019). Naturally, this led to developments in automated calcification segmentation algorithms using medical images, as further discussed in Section 2.

Calcification is also crucial for accurate biomechanical simulations. When modeling the valve leaflet dynamics, calcification can induce stiffer material properties and cause irregular stress distributions (Qin et al., 2020). Similarly, for Transcatheter Aortic Valve Replacement (TAVR) simulations, the high-stress regions of interest often correspond to regions with high calcification (Wang et al., 2015). Past studies have thus incorporated calcification into their simulations using fully or semi-automated calcification segmentation (Grbic et al., 2013; Morganti et al., 2014).

The caveat is that most calcification segmentation algorithms output a voxelgrid representation, whereas biomechanics methods most commonly require a mesh representation. Often times, extensive manual work and/or error-prone mesh editing operations are required to convert the segmentation into a final desired geometry.

For calcified heart meshing, we must consider two main components with substantially different geometrical characteristics. First is the native heart tissue, which is more “regular” and has a relatively consistent topological structure across patients. Second is the calcification, which is much more “irregular” and has a highly variable volume, positioning, and topology. Due to these characteristics, heart meshing has been widely studied using template deformation methods (Kong et al., 2021; Kong and Shadden, 2022; Pak et al., 2023), but robust calcification meshing still remains a significant challenge. This issue also applies to other medical imaging tasks that aim to model both “regular” and “irregular” objects, such as anatomical tissues combined with tumors, infarct regions, and other visible abnormalities.

To our knowledge, there has been little to no prior work on robust automated meshing of “irregular” objects that also establishes strong physical relations to the surrounding geometry. We aim to provide a solution for this with the following contributions:
1. Segmentation algorithms that encourage better anatomical consistency between the predicted calcification and the neighboring heart structures
2. A robust calcification meshing algorithm that establishes complete contact surface matching to a given heart mesh without affecting the original heart mesh topology
3. Mesh viability checks for solid mechanics simulations of valve opening and TAVR stent deployment

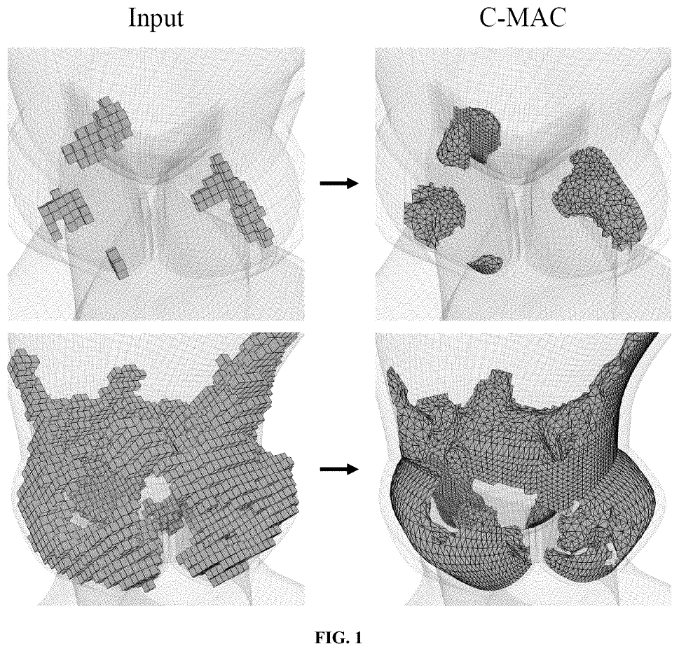

Together, these contributions amount to an end-to-end framework for robust automated calcification meshing from CTA that complements previous works in heart geometry reconstruction and calcification segmentation. Our algorithm drastically improves both the success rate and the resulting element quality of the final volume meshing steps. Additionally, our approach enables large-scale simulations by preserving the inter-patient mesh correspondence of the original heart geometry. Sample results demonstrating our task and method performance are shown in FIG. 1.

### 2. BACKGROUND AND RELATED WORKS

For most biomechanics studies, we are interested in the effects of calcification on its tissue-of-origin. In this study, we focused on the TAVR-relevant heart structures—namely, the aortic valve, ascending aorta, and left ventricular myocardium. This allowed us to validate our calcification meshing approach with aortic valve-related solid mechanics simulations.

Among the listed heart structures, the aortic valve (i.e. the aortic root wall plus the aortic valve leaflets) is arguably the hardest object to reliably reconstruct using traditional segmentation methods on CTA due to the limited image resolution, complex geometry, and calcification-induced imaging noise. Studies have thus specifically tackled voxelgrid segmentation of the aortic valve using multi-atlas and deep learning (DL) methods (Pouch et al., 2013; Pak et al., 2020). The resulting segmentation can be converted to shape models using posthoc algorithms, such as medial modeling and shape registration (Pouch et al., 2015; Astorino et al., 2012). Alternatively, others have proposed directly fitting a template mesh based on handcrafted image features and boundary detectors (Ionasec et al., 2010; Weese et al., 2017). Recently, deep learning-based template deformation methods were proposed for direct image-to-mesh aortic valve modeling (Pak et al., 2021a,b). We used the expanded version of this approach (Pak et al., 2023) to generate the initial heart mesh to which we applied our calcification modeling algorithms.

For aortic calcification segmentation, both non-contrast CT and contrast-enhanced CTA have been explored as inputs. Global thresholding on non-contrast CT and filtering using aorta segmentation is one possible direction (Kurugol et al., 2015), but most other studies focused on calcification segmentation using CTA. Adaptive thresholding based on luminal attenuation is the most common approach (Mahabadi et al., 2009; Alqahtani et al., 2017; Bettinger et al., 2017; Vlastra et al., 2019). Learning-based methods have also been proposed using hand-crafted features (Grbic et al., 2013; Harbaoui et al., 2016) and deep learning (Graffy et al., 2019). We took inspiration from these prior works and obtained the calcification segmentation in two steps. We first predicted the initial segmentation using a deep learning model, and then applied a simple postprocessing technique to greatly improve its anatomical consistency with the heart geometry.

Incorporating calcification into TAVR simulations has been achieved through various means, such as assigning different element properties on the native tissue geometry (Morganti et al., 2014), defining surface-based tie constraints Russ et al. (2013), re-meshing with a nearby tissue (Bianchi et al., 2019), and manual stitching (Wang et al., 2015). For some, this process was left elusive, described simply as a finite element (FE) modeling process from segmentation (Grbic et al., 2013). We propose an automatic calcification meshing technique that ensures robust node connections to the surrounding tissue geometries. This holds several advantages over previous approaches, such as (1) incorporation of the non-negligible calcification geometry, (2) more explicit and robust node constraints instead of surface-based tie constraints, (3) ability to maintain the original tissue mesh topology, and (4) automated workflow with no expert-driven manual adjustments.

To accomplish this, we took inspiration from existing meshing algorithms, particularly those that use voxelgrid signed distance functions (SDF) as inputs. Within this context, marching cubes is one of the most popular surface meshing algorithms that tessellates the input space defined by a cubic lattice (Lorensen and Cline, 1987). Despite its many strengths, marching cubes can result in topological inconsistencies with respect to the original input due to ambiguities associated with cubic tessellation. Marching tetrahedra addresses this by tessellating a tetrahedralized cubic lattice instead (Payne and Toga, 1990), which has been further refined through body-centered cubic lattice (Chan and Purisima, 1998) and pre-triangulation vertex clustering Treece et al. (1999). More recently, Deep Marching Tetrahedra (DMTet) was introduced as a way learn the mesh generation process (Shen et al., 2021). The method primarily entails utilizing various deep learning architectures to learn (1) the deformation of the background tetrahedral lattice and (2) the associated SDF values at each background mesh vertex.

As suggested by the extensions of marching tetrahedra, the background mesh has a crucial effect on the final output mesh. With a regular lattice, it is difficult to control the exact output mesh topology, which necessitates suboptimal post-processing for establishing node correspondence with an existing mesh. Pak et al. (2023) ran into this exact issue with calcification meshing, where the authors used a regular lattice isosurface extraction followed by a simplified distance-based node projection, which lead to inconsistent node correspondence and some heavy element distortions. Therefore, instead of relying on a pre-defined regular lattice, we leveraged boundary-constrained mesh generation techniques to construct our background grid. TetGen is a popular choice for this task due to its robust performance and widely available code (Hang, 2015).

### 3. METHODS

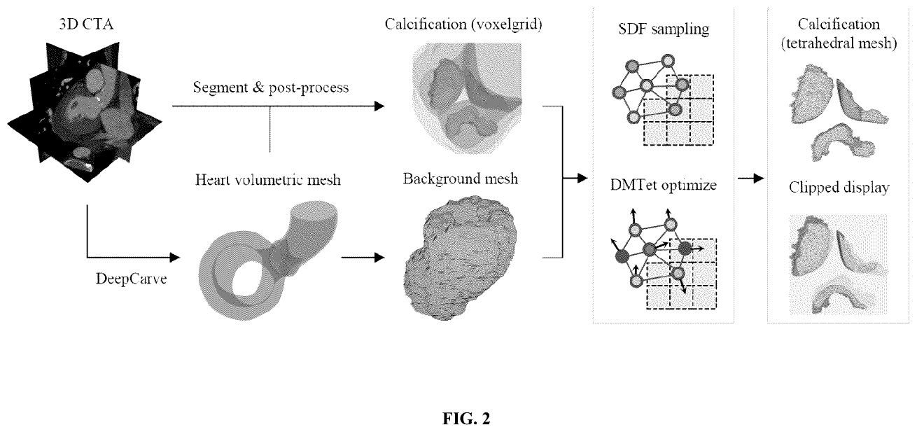

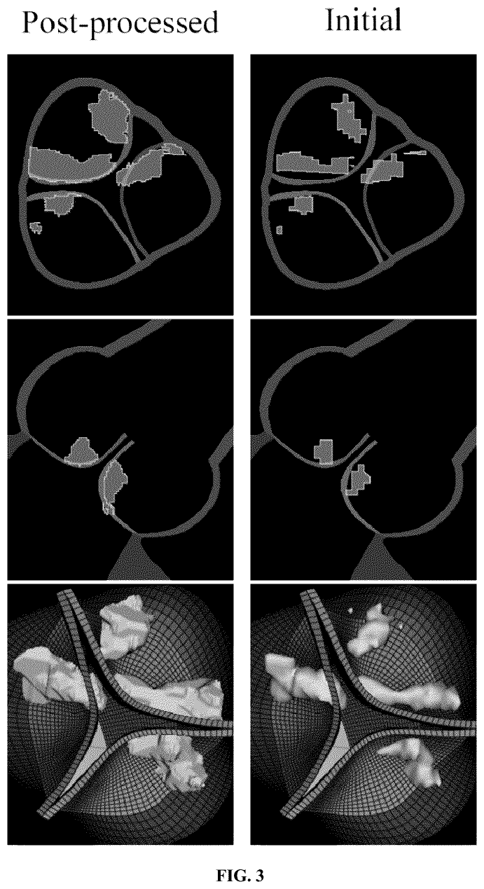

The overarching workflow of C-MAC is presented in FIG. 2 and Alg. 1. The main goal is to reconstruct the calcified heart geometry for structural simulations such as TAVR and valve opening. To enable large-scale analyses, we sought to maintain the inter-patient mesh correspondence of the heart geometry. We split the problem into three sub-tasks: heart geometry reconstruction, calcification segmentation, and calcification meshing.

### 3.1. Heart Geometry Reconstruction

We used DeepCarve (Pak et al., 2023) for heart geometry reconstruction with a few minor adjustments to the training strategy. Here, we briefly describe the method for completeness.

Algorithm 1 C-MAC overview

| | |
|---|---|
| 1: | function C-MAC(I) |
| 2: | Mₕₑₐᵣₜ ← DeepCarve(I) |
| 3: | y_{ca2} ← Ca2Segmentation(I, Mₕₑₐᵣₜ) |
| 4: | M_{ca2} ← Ca2Meshing(y_{ca2}, Mheart) |
| 5: | return (Mₕₑₐᵣₜ, M_{ca2}) |

I: image,
y: segmentation,
M: volume mesh,
ca2: calcification

The goal is to train a deep learning model that will take an input image and generate a patient-specific mesh by deforming a template mesh. The required training label is a set of component-specific pointclouds that can help measure the spatial accuracy of the prediction. The predicted mesh is penalized for large deformations from the original template elements via deformation gradient-based isotropic and anisotropic energies. The deformation generated by the model is constrained to be a diffeomorphic b-spline field to enforce additional smoothness. For additional detail, please refer to the original publication.

The three minor modification for this work are (1) random translation augmentation, (2) a better LV template with fewer distorted elements, and (3) the addition of 0.1*AMIPS (Fu et al., 2015) to the training loss.

Once DeepCarve is trained, a single forward-pass directly generates volumetric meshes of the heart geometry with high spatial accuracy and element quality. In this work, we assumed accurate heart geometry reconstruction and focused on robustly incorporating calcification to the resulting output.

### 3.2. Calcification Segmentation

Voxelgrid segmentation is useful for modeling sporadic structures like calcification. Our contributions for this specific task are (1) a deep learning strategy for fully automating the initial segmentation and (2) a post-processing method that encourages anatomical consistency between the final segmented calcification and the predicted heart geometry (Alg. 2).

Algorithm 2 Ca2 segmentation

| | |
|---|---|
| 1: | function Ca2Segmentation(I, Mₕₑₐᵣₜ) |
| 2: | y₀ ← DLCa2Seg(I) |
| 3: | y_{ca2} ← PostProcessCa2Seg(y₀, Mₕₑₐᵣₜ) |
| 4: | return y_{ca2} |

### 3.2.1. Initial Segmentation Via Deep Learning

(DLCa2Seg) Trained deep learning models can output an initial calcification segmentation with a single forward-pass of the target image. For our task, our model needs to be able to handle:
Highly unbalanced target (i.e. calcification is very sparse compared to the input image volume)
Empty target (i.e. no calcification in the input image)
Accurate segmentation boundary

To meet these criteria, we used a modified 3D U-net (Ronneberger et al., 2015) and tested four different training strategies involving the following losses:
1. Dice+Cross Entropy (DiceCE)
2. Generalized Dice Loss (GDL)
3. GDL+boundary loss (Gbd)
4. GDL+chamfer loss (Gch)

Dice (a.k.a. DSC) (Drozdzal et al., 2016; Sudre et al., 2017) and cross entropy (CE) (Ronneberger et al., 2015) are commonly used for training segmentation models. Dice has several advantages over CE, such as being more robust to unbalanced targets and being able to directly optimize a common evaluation metric, but it is ineffective at handling empty targets. A common workaround is to instead optimize the combined DiceCE loss (Ma et al., 2021), which can be defined for binary segmentation tasks as

$${ℒ}_{DiceCE} ( y , \hat{y} ) = 1 - 2 \frac{{\sum }_{i = 1}^{N} {y}_{i} {\hat{y}}_{i}}{{\sum }_{i = 1}^{N} {y}_{i} + {\sum }_{i = 1}^{N} {\hat{y}}_{i} + ϵ} + λ [ \frac{1}{N} ( {\sum }_{i = 1}^{N} {y}_{i} log ( {\hat{y}}_{i} ) + ( 1 - {y}_{i} ) log ( 1 - {\hat{y}}_{i} ) ) ] \qquad ( 1 )$$

where yᵢ is the target and ŷᵢ is the predicted value at each i^{th} voxel, λ is a weighting hyperparameter, and ϵϵ is an error term that prevents division by 0. To prevent numerical issues with log(0), the log output is set to have a minimum bound at −100 (Paszke et al., 2017).

Generalized Dice Loss (GDL) is a variation of the Dice loss that handles multi-class segmentation with a per-class weighting term that mitigates biases towards larger segments (Sudre et al., 2017). For a binary segmentation task, we can treat the background as its own class, in which case

$${ℒ}_{GDℒ} ( y , \hat{y} ) = 1 - 2 \frac{{\sum }_{c = 1}^{2} {w}_{c} {\sum }_{i} {y}_{ci} {\hat{y}}_{ci}}{{\sum }_{c = 1}^{2} {w}_{c} {\sum }_{i} ( {y}_{ci} + {\hat{y}}_{ci} )} \qquad ( 2 )$$

where

$${w}_{c} = \frac{1}{{( {\sum }_{i} {y}_{ci} + {ϵ}_{c} )}^{2}} .$$

We set ∈₀=−100 and ∈₁=100 via hyperparameter tuning, where c=0 is background and c=1 is calcification. The ∈_{c} helps prevent division by 0 and helps maintain a smooth Dice-like behavior for both empty and sparse targets. Empirically, GDL performed better than DiceCE for our task, so it was chosen as the main region-based loss for the rest of our experiments.

All variants of distribution-based losses (i.e. CE) and region-based losses (i.e. Dice) penalize each voxel prediction independently, and fail to capture the magnitude of error from the original segmentation boundary. The boundary loss was proposed to combat this phenomenon (Kervadec et al., 2019):

$${ℒ}_{Gbd} ( y , \hat{y} ) = {ℒ}_{GDL} ( y , \hat{y} ) + λ {\sum }_{i} - {SDF ( y )}_{i} {\hat{y}}_{i} \qquad ( 3 )$$

where SDF(y) is the signed distance function (SDF) calculated using the ground-truth segmentation y. By this definition, SDF is assumed to be positive on the inside and negative on the outside of the y=0.5 isosurface. The weighting hyperparameter λ is adjusted based on training epochs, similar to Kervadec et al. (2019). In our case, λ=0 at epoch 0 and was linearly increased to λ=1000 from epochs 1000-2000. We clipped the SDF from −3 to 3 for numerical stability.

Finally, instead of relying on the integral approximation approach as did Kervadec et al. (2019), we evaluated the effectiveness of directly optimizing for the symmetric chamfer distance (Wang et al., 2018) between the predicted and target segmentations:

$${ℒ}_{Gch} ( y , \hat{y} ) = {ℒ}_{GDL} ( y , \hat{y} ) + λ ( \frac{1}{\vert A \vert} {\sum }_{a ϵ A} {min}_{b ϵ B} {‖ a - b ‖}_{2}^{2} + \frac{1}{\vert B \vert} {\sum }_{b ϵ B} {min}_{a ϵ A} {‖ b - a ‖}_{2}^{2} ) \qquad ( 4 )$$

where A and B are pointclouds sampled on the segmentations' extracted surfaces. We used DMTet to extract the surface from the predicted segmentation, which makes the operation differentiable with respect to the segmentation model parameters (Shen et al., 2021). λ=0 at epoch 0 and linearly increased to λ=0.1 from epochs 1000-2000.

All models were trained for 4000 epochs with the Adam optimizer, at a learning rate of 1e-4. For data augmentation, we used random translation and b-spline deformation.

We also compared our models' performance against nnUnet, a popular self-configuring DL segmentation method. For fair comparisons, we trained the nnU-net with the same training/validation/test splits as our own models, which effectively discarded nnU-net's default ensembling operations.

### 3.2.2. Post-Processing for Anatomical Consistency

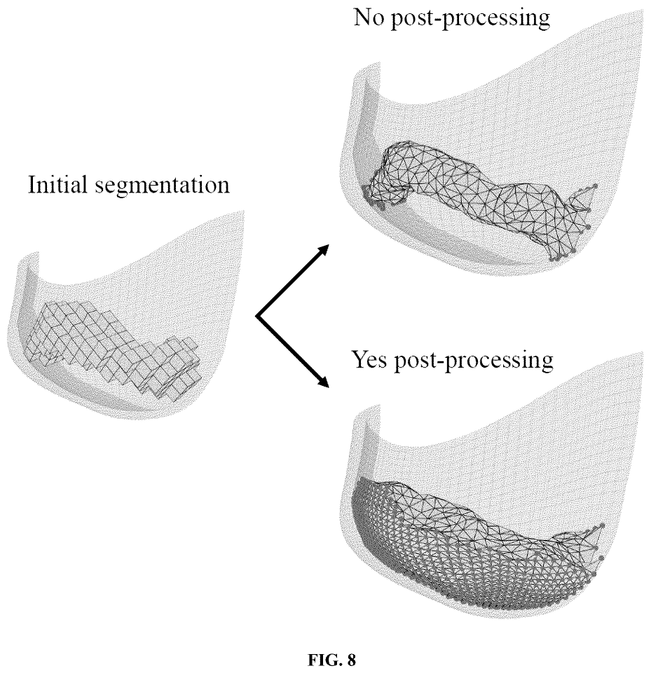

We propose an effective automated post-processing method that greatly improves the anatomical consistency of the final calcification segmentation (Alg. 3). In our task, this means removing the undesired spatial gaps between the calcification segmentation and the heart mesh (FIG. 3). Without this step, the predicted segmentation is usually only a few voxels away from the heart geometry, but that could still significantly affect the quality of the final reconstructed meshes, especially along the contact surfaces (FIG. 8).

Algorithm 3 Post-processing of ca2 segmentation

| 1: | function PostProcessCa2Seg(y₀, Mₕₑₐᵣₜ) | |
|---|---|---|
| 2: | {yᵢ} ← GroupSegByLeaflets(y₀) | [symbol] i ϵ {1,2,3} |

| | |
|---|---|
| 3: | while not satisfied do |
| 4: | for yᵢ in {yᵢ} do |
| 5: | yₕₑₐᵣₜ ← FilterHeartSeg(yᵢ, Mₕₑₐᵣₜ) |
| 6: | yᵢ ← AdaptiveClose(yᵢ, Ŝₕₑₐᵣₜ) |
| 7: | y_{ca2} ← SubtractAndFilterSeg({yᵢ}, Mₕₑₐᵣₜ) |
| 8: | return y_{ca2} |

Sᵢ is assigned in-place, so Sᵢ is updated with each iteration.

Our main method for filling in the gaps is morphological closing (dilation→erosion). Unfortunately, a naïve application of this approach leads to overly connected components, such as undesired calcification fusing and excessive contact with the neighboring heart structures. We have added several operations to minimize the deviation from the initial segmentation while still being able to fill in the gaps.

(GroupSegByLeaflets) First, we split the calcification segmentation into three groups—one group for each aortic valve leaflet. Each island of the segmentation was assigned to the closest aortic valve leaflet, measured by the mean symmetric chamfer distance.

(FilterHeartSeg) Using the grouped calcification segments and the predicted heart mesh, we extracted heart segments that are within some distance away from the calcification. We first converted the heart mesh to heart segments using the image stencil operation (Schroeder et al., 1998), and only kept the segments intersecting with dilated calcification segments. The dilation filter was 7×7×7 voxels and adaptive, meaning the filter values were ellipsoidal with the major principal axis pointing in the same direction as the closest heart mesh node's surface normal.

(AdaptiveClose) We used a two-step morphological close operations on the grouped calcification segments and the corresponding filtered heart segments. The first closing operation was performed with the same adaptive kernel described in FilterHeartSeg, and the second closing operation was performed with an isotropic spherical 3×3×3 filter.

(SubtractAndFilterSeg) Due to the limited resolution of the voxelgrid representation, directly meshing from the combined segments results in many undesired protrusions in the final mesh. To avoid this, we filtered the combined segments by subtracting away the heart segments and performing volume-based island removals. For the subtraction, we tripled the segmentation resolution to make sure that we can capture the thin leaflet geometries in the voxelgrid representation. The final segmentation for downstream meshing (yca2) therefore has a 3× voxelgrid resolution compared to the original image.

### 3.3. Calcification Meshing

Algorithm 4 Meshing for complete contact surface matching

| | |
|---|---|
| 1: | function Ca2Meshing(y_{ca2}, Mₕₑₐᵣₜ) |
| 2: | M_{bg}← GenerateBackgroundMesh(Mₕₑₐᵣₜ) |
| 3: | S_{ca2}← DMTetOpt(y_{ca2}, M_{bg}) |
| 4: | if not satisfied then |
| 5: | S_{ca2}← ConstrainedRemesh(S_{ca2}) |
| 6: | M_{ca2} ← TetGen(S_{ca2}) |
| 7: | return M_{ca2} |

M: volume mesh,
S: surface mesh,. [symbol]
: uniform distribution

(DMTet) Deep Marching Tetrahedra (Shen et al., 2021) is at the core of our calcification meshing algorithm. DMTet outputs an isosurface triangular mesh given a background tetrahedral mesh and its nodal SDF. It defines the typology of the isosurface based on the sign changes of the SDF and the location of the isosurface based on the magnitude of the SDF (Eq. 5).

$${v}_{ab}^{′} = \frac{{v}_{a} \cdot SDF ( {v}_{b} ) - {v}_{b} \cdot SDF ( {v}_{a} )}{SDF ( {v}_{b} ) - SDF ( {v}_{a} )} \qquad ( 5 )$$

Here, vᵢ is the nodal position and SDF(vᵢ) is the nodal SDF. SDF(vₐ)≤0 and SDF(v_{b})>0. Note that the node interpolation is only performed along edges where there is a “sign” change, i.e. between a positive SDF node and a non-positive SDF node. This prevents division by 0.

Under this formulation, we found that the best way to ensure complete contact surface matching is to optimize both the background mesh and the nodal SDF. Following DMTet optimization, we optionally remeshed the non-contact surfaces to reduce the element density for faster simulations. The details of each step are described in the subsequent subsections.

The final surface meshes were converted to tetrahedral meshes via TetGen and merged with the predicted heart mesh at coincident nodes (distance<1e-3) (Alg. 4).

### 3.3.1 Background Mesh Generation

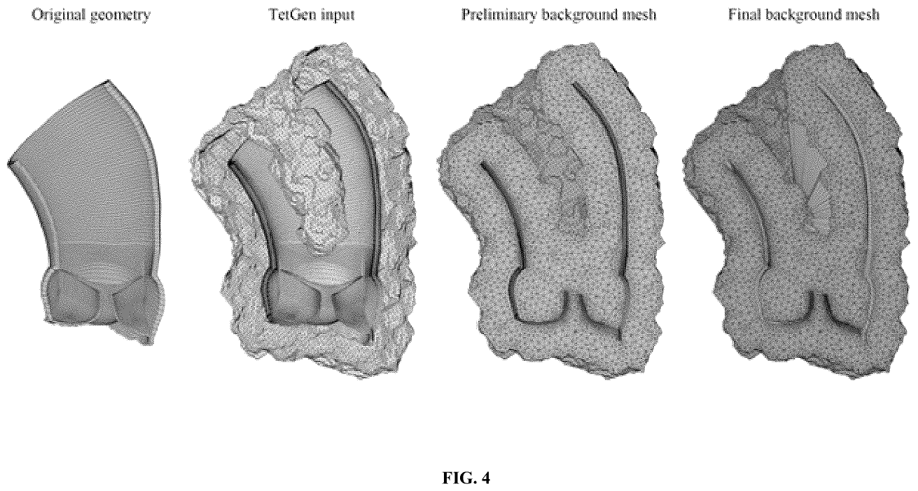

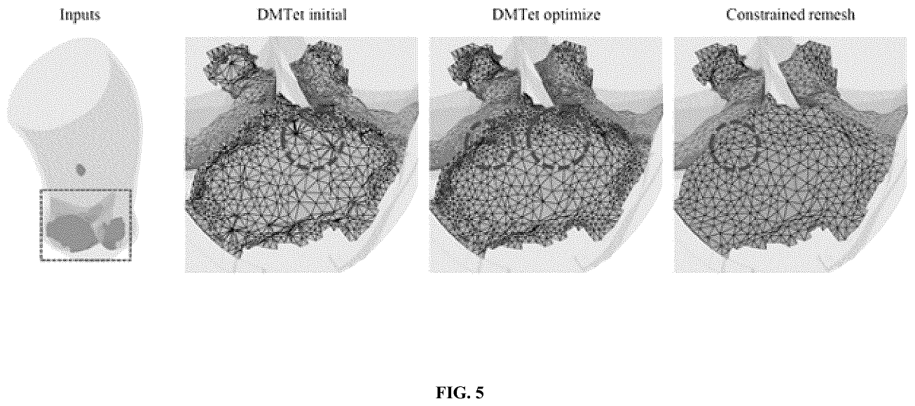

The background mesh generation is summarized in FIG. 4 and Alg. 5. The main goal was to generate a tetrahedral background mesh that includes the original heart surfaces.

Algorithm 5 Background mesh generation

```
function GenerateBackgroundMesh(Mₕₑₐᵣₜ)
  Sₐₒᵣₜₐ← ExtractAortaSurf(Mₕₑₐᵣₜ)
  S_{offset}← OffsetSurf(Mₕₑₐᵣₜ)
  Mₚᵣₑₗᵢₘ ← TetGenAndHollow(Sₐₒᵣₜₐ, S_{offset})
  M_{bg} ← CreateFakeElems(Mₚᵣₑₗᵢₘ)
  return M_{bg}
```

(ExtractAortaSurf) Since we were focusing on aortic calcification, the original heart surface consisted of the surface elements of the aorta and the aortic valve leaflets, which we can extract using standard meshing libraries (Schroeder et al., 1998).

(OffsetSurf) For the outer bound of the background mesh, we generated an offset surface that includes all areas within 10 voxel spacings away from the aortic surface. The extracted aortic surface and the offset surface were combined by addition, which was easily doable because the offset surface and the extracted heart surface are clean manifold surfaces with no intersections by construction.

(TetGenAndHollow) The merged surfaces were processed by TetGen for constrained tetrahedral meshing, and then the elements inside the aortic surface were removed.

(CreateFakeElems) Lastly, we created “fake” tetrahedral elements by adding a “fake” node to all background boundary surfaces, i.e. the aortic surface and the offset surface elements.

TetGenAndHollow and CreateFakeElems are crucial for accurate meshing. The rationale will be explained further in the following subsection.

### 3.3.2 DMTet Optimization

We used DMTet to convert the post-processed voxelgrid segmentation to a surface mesh. The DMTet optimization process is outlined in Alg. 6.

Algorithm 6 DMTet optimization

```
function DMTetOpt(y_{ca2}, M_{bg})
  (V_{bg}, E_{bg}) ←M_{bg}
  δ~ [symbol]   ₙ(−1e − 3,1e − 3)
  for nₒₚₜ do
    (ΔV_{bg}, ΔSDF) ← δ
    \tilde{V}_{bg}← V_{bg} + ΔV_{bg}
    \tilde{M}_{bg}← (V_{bg}, E_{bg})
    SDF ← LinearMapAndInterp(y_{ca2}, \tilde{V}_{bg})
    SDF ← ModifyEdgeCases(SDF) [symbol]
    ← SDF + ΔSDF
    S_{DMTet} ← DMTet( [symbol]   , \tilde{M}_{bg})
    (V, E) ← S_{DMTet}
    δ ← Adam (δ, [symbol]   _{DMTet}(V, E, ΔV_{bg}))
  return S_{DMTet}, [symbol]   _{DMTet}, δ
```

(LinearMapAndlnterp) We first converted the predicted segmentation to a simplified voxelgrid SDF via linear mapping SDF(ŷ)=2ŷ−1, which means ŷᵢ∈ {0,1}→SDF(ŷ)ᵢ ∈{−1,1}. Then, we trilinearly interpolated the SDF at each background mesh node. To demonstrate the need for further processing, let us consider applying marching tetrahedra with the default SDFᵢ∈{−1,1} and the preliminary background mesh. Due to the smooth transition of the nodal SDF magnitudes, none of the resulting surface mesh would match exactly with the original background mesh nodes. This is counterproductive, as our desired output is a surface mesh with coincident nodes and edges along the contact surfaces.

(ModifyEdgeCases) To enforce complete contact surface matching, we make two modifications to the nodal SDF after the initial interpolation:

$${SDF}_{i} = \{ \begin{matrix}0 & if background boundary node \\ - 1 e 12 & if fake node \\ {SDF}_{i} & else\end{matrix} \qquad ( 6 )$$

Then, from Eq. 5, this results in the following node assignments:

$${v}_{ab}^{′} \approx \{ \begin{matrix}{v}_{a} & if background boundary node \\ {v}_{b} & if fake node \\ {v}_{ab}^{′} & else\end{matrix} \qquad ( 7 )$$

Recall that (1) all elements inside the heart volume were removed via TetGenAndHollow and (2) the fake node was added to the background boundary surface to generate fake tetrahedral elements via CreateFakeElems (FIG. 4). Together with Eq. 7, this means that any SDF sign change involving a boundary node will always result in that node being included as a part of the DMTet output. This is exactly our intended output with complete contact surface matching with the original boundary elements.

Applying Eq. 6 is enough for establishing complete contact surface matching. However, another known problem of marching tetrahedra is the irregular elements of the output surface (Treece et al., 1999). Although some related solutions exist, our task also requires that the contact surfaces be preserved during mesh processing. DMTet optimization is the first step of our two-part solution. Constrained surface remeshing is the optional second step (Section 3.3.3).

Similar to the original DMTet (Shen et al., 2021), we optimized (1) the deformation of the background mesh and (2) the offset from the initial interpolated nodal SDF. Unlike the original paper, we do not train a deep learning model for the prediction. Instead, we perform the optimization for each inference target independently. The rationale for this choice is described in Section 5. During the optimization process, we minimize the output's deviation from the original segmentation by penalizing the background mesh deformation and prohibiting sign changes in the nodal SDF.

The overall idea is to improve the mesh quality while minimizing its deviation from the original segmentation. The loss is a combination of Laplacian smoothing, edge length, edge angle, and deformation penalty (Eq. 8). The first three losses help improve the overall mesh and individual element qualities, while the deformation penalty helps minimize surface shrinking. Eq. 6 is applied after each optimization step, so only the non-contact nodes' SDF values are optimized during this process.

$${ℒ}_{DMTet} ( V , E , Δ {V}_{bg} ) = {λ}_{0} {\sum }_{{v}_{i} ∈ V} {‖ \frac{1}{\vert N ( {v}_{i} ) \vert} {\sum }_{{v}_{j} ∈ N ( {v}_{i} )} {v}_{i} - {v}_{j} ‖}_{2} + {{λ}_{1} [ {\sum }_{{v}_{i} ∈ V} {\sum }_{{v}_{j} ∈ N ( {v}_{i} )} {( {‖ {v}_{i} - {v}_{j} ‖}_{2} - ϵ )}^{2} ]}^{\frac{1}{2}} + {{λ}_{2} [ {\sum }_{a ∈ A ( V , E )} {( a - α )}^{2} * σ ( a ) ]}^{\frac{1}{2}} + {λ}_{3} {\sum }_{{v}_{i} ∈ {V}_{bg}} {‖ Δ {v}_{i} ‖}_{2} \qquad ( 8 )$$

(V,E): vertices and edges of the DMTet output. N: neighboring nodes. A: angles between all edges. ϵ: desired edge length, set to 0.1 for our task. α: desired edge angle, set to 60 degrees. λᵢ: hyperparameters, each set to [10, 1, 1, 0.5]. σ(a)=(1−sigmoid(a−10))+sigmoid(150): filter to minimize the penalty for “good enough” angles, i.e. between 10 and 150 degrees.

We used the Adam optimizer with a learning rate of 1e-2 and achieved convergence at around nₒₚₜ=100. For the final DMTet output, we cleaned the mesh by removing overlapping nodes (distance<1e-2) and elements with repeated nodes (i.e. points and edges), which are caused by Eq. 6.

### 3.3.3 Constrained Surface Remeshing

Although the DMTet output is already a suitable manifold surface for the final tetrahedralization, it can be further refined via surface remeshing (Alg. 7). The main remeshing algorithm we used is ACVD, a Voronoi diagram-based vertex clustering method (Valette et al., 2008).

Algorithm 7 Constrained remeshing by vertex-clustering

```
function ConstrainedRemesh(S_{ca2})
  for nᵣₑₘₑₛₕ do
    S_{contact}, S_{free}← SeparateContactSurf(S_{ca2})
    S_{free2} ← ConstrainedClustering(S_{free})
    S_{ca2} ← Merge(S_{contact}, S_{free2})
  return S_{ca2}
```

To preserve the contact surface elements, we made two modifications to the remeshing steps.

(SeparateContactSurf) First, we split the initial surface into contact and non-contact elements by checking the number of nodes merged with the original heart mesh's nodes. If all three nodes of a triangular element are coincident with heart mesh nodes, then that element is a contact element.

(ConstrainedClustering) We performed the initial ACVD clustering step on the non-contact surface, which assigns a cluster index to each node. The number of initial clusters was set to 80% of the number of nodes. The nodes belonging to both the contact and non-contact elements were preserved by assigning new unique cluster indices. Note that the percentage of vertices actually being clustered progressively decreases with each remeshing step because of our cluster re-assigning scheme. Finally, we performed the standard ACVD triangulation to obtain the remeshed non-contact surface.

(Merge) We merged the contact surfaces and the remeshed non-contact surfaces to obtain the final output surface mesh. This step was easy to perform because we preserved the coincident nodes between the contact and non-contact surfaces using constrained clustering.

The constrained remeshing was repeated as many times as necessary to achieve the desired element size and density. We often stopped at nᵣₑₘₑₛₕ=15 to prevent oversimplification. The remeshing step is optional, as we can obtain good quality tetrahedral calcification meshes directly from the output of DMTetOpt. However, we found that the remeshing step generally further improves the element quality for downstream applications.

### 3.3.4 Final Tetrahedralization

We obtained the final tetrahedral calcification mesh by applying TetGen on the surface mesh from the previous subsections. This step was straightforward because we designed the surface meshes to be clean and manifold. For node-stitching, we first assigned each connected component of calcification to a leaflet based on the lowest mean symmetric chamfer distance, and nodes from the assigned leaflet or the aortic wall were merged with calcification nodes if the distances were less than 1e-3. Connected components of calcification that had no merged nodes with heart surfaces were removed.

### 4. RESULT

### 4.1 Data Acquisition and Preprocessing

We used the same dataset as DeepCarve with some minor modifications. The original dataset consisted of 80 CTA scans and the corresponding labels for the heart structures and calcification. 14 of the 80 CT scans were from 14 different patients in the training set of the MM-WHS public dataset Zhuang and Shen (2016). The remaining 66 scans were from 55 IRB-approved TAVR patients at Hartford hospital. We used more than one time point for some of the Hartford patients. Of the 80 total scans, we used 35/10/35 scans for training/validation/testing. We ensured that the testing set had no overlapping patients with the training/validation sets. All evaluations were performed with the 35 test-set cases. All patients had tricuspid aortic valves and varying levels of calcification. Some scans had no aortic calcification.

The calcification segmentation was obtained using adaptive thresholding and manual post-processing. The original dataset included calcification that is within some distance away from any of the pointclouds. For this work, we further processed it to only include segments around the aorta. This was mainly to omit calcification along the mitral valve, which would require accurate mitral valve leaflet geometry for us to properly apply our meshing techniques.

Both Gbd and Gch require extra training labels extracted from the original calcification segmentation. Gbd requires voxelgrid SDF, which we obtained using isosurface extraction and point-to-mesh distance from each voxel position. Gch requires calcification pointclouds, which we obtained using isosurface extraction and surface point sampling.

For preprocessing, we rescaled all images and labels to an isotropic spatial resolution of 1.25 mm3 and cropped them to 1283 voxels. The default crop center was the center of the ground-truth heart pointclouds' bounding box. For translation augmentation, the crop center was offset by a random sample from a 3D Gaussian (stdev=crop width/3), and the offset amount was capped to prevent any labeled structures being outside the cropped region. The images were not pre-aligned with any additional transforms.

Image intensities were first clipped to [lower bound, upper bound], and further min-max normalized to [0,1]. The lower and upper bounds were fixed for DeepCarve at [−158,864]Hounsefield Units (HU) and chosen between three different ranges for calcification segmentation, as specified in the results tables (Table. 1, 2).

### 4.2 Deep Learning Segmentation Evaluation

For the initial calcification segmentation, we mainly focused on finding the optimal training strategy for our task-specific deep learning model. The evaluations are summarized in Table 1. We first observed that nnU-net Isensee et al. (2021), a popular self-configurable segmentation algorithm, suffers from a high rate of false positive segments outside of the aorta. All of our methods had better overall performance than nnU-net. The only metric that nnU-net performed relatively well was the number of false positives for empty targets (i.e. when there is no aortic calcification in the CTA scan).

Among our own models, we first compared the effects of different image intensity ranges for the min-max normalization, while using the DiceCE and GDL losses. The metrics generally indicate that (1) using the right intensity range can help boost the performance and (2) GDL with I minmax [−200,1500] achieves the best overall performance.

Then, we used the best performing training setup from our initial evaluation and tested the modified GDL losses: Gbd and Gch. Gbd was effective at reducing false positives, but otherwise both Gbd and Gch showed worse overall performance than the standard GDL loss. For our task, false positives for empty targets were less critical than accurate prediction of non-empty targets because false positives for empty targets can be easily identified and omitted using the source image. Therefore, we used the GDL with I minmax [−200,1500] as our final segmentation model for downstream tasks.

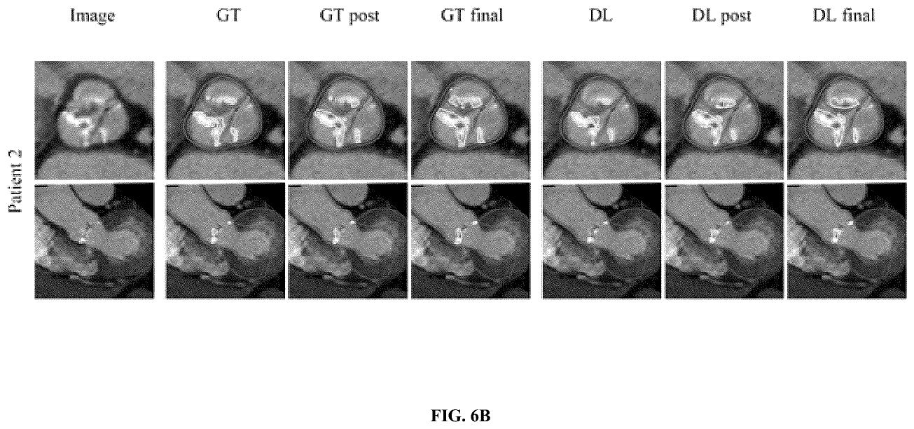

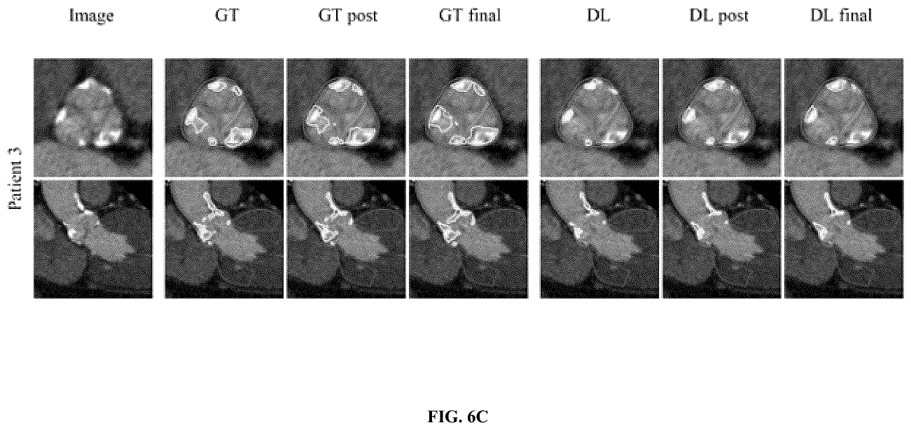

FIGS. 6A-C contain some examples of ground-truth segmentation vs. DL segmentation using “GDL (ours)” (column 2 vs. column 5). Other than Patient 3 slice 1 (row 5), the DL segmentation qualitatively matches the ground-truth very well.

TABLE 1
Deep learning model performances on the initial
calcification segmentation (DLCa2Seg).

| Experiment condition | | Non-empty target segmentation | | | | Empty target |
|---|---|---|---|---|---|---|
| Train | I | | HD 95% | CD | CD_{Heart} | False pos |
| strategy | minmax | Dice ↑ | (mm) ↓ | (mm) ↓ | (mm) ↓ | (vx) ↓ |
| nnU-net | self-configured | 0.656 ± | 13.259 ± | 2.337 ± | 2.501 ± | 5.4 ± |
| | | 0.222 | 18.055 | 2.994 | 2.955 | 10.0 |
| DiceCE | [−158, 864] | 0.685 ± | 8.254 ± | 1.462 ± | 1.345 ± | 25.5 ± |
| | | 0.180 | 11.605 | 2.421 | 0.319 | 25.2 |
| DiceCE | [−200, 1500] | 0.690 ± | 8.362 ± | 1.320 ± | 1.653 ± | 28.9 ± |
| | | 0.144 | 13.141 | 1.653 | 0.922 | 30.1 |
| DiceCE | [−1000, 2500] | 0.680 ± | 5.684 ± | 1.155 ± | 1.409 ± | 151.9 ± |
| | | 0.134 | 8.165 | 1.529 | 0.301 | 249.2 |
| GDL | [−158, 864] | 0.685 ± | 6.503 ± | 1.380 ± | 1.381 ± | 48.9 ± |
| | | 0.184 | 9.066 | 2.102 | 0.419 | 81.0 |
| GDL (ours) | [−200, 1500] | 0.709 ± | 4.668 ± | 0.842 ± | 1.314 ± | 31.5 ± |
| | | 0.111 | 6.977* | 1.081* | 0.280* | 44.2 |
| GDL | [−1000, 2500] | 0.679 ± | 7.336 ± | 1.419 ± | 1.711 ± | 129.2 ± |
| | | 0.152 | 9.745 | 1.932 | 1.320 | 171.6 |
| Gbd | [−200, 1500] | 0.675 ± | 5.797 ± | 1.000 ± | 1.319 ± | 4.4 ± |
| | | 0.142 | 7.180 | 1.002 | 0.295 | 4.8 |
| Gch | [−200, 1500] | 0.672 ± | 6.306 ± | 1.098 ± | 1.365 ± | 16.2 ± |
| | | 0.150 | 8.282 | 1.089 | 0.306 | 14.3 |

I minmax: range for image intensity min-max normalization,
HD: Hausdorff distance,
CD: mean symmetric chamfer distance,
CDHeart: one-sided chamfer distance from calcification to heart surfaces,
False pos: false positives,
mm: millimeters,
vx: voxels,
*p < 0.05 between GDL (ours) and nnU-net. All metrics are mean ± stdev across patients.

TABLE 2
Segmentation metrics after applying our post-processing algorithm (PostProcessCa2Seg)
on each representative segmentation prediction model.

| Experiment condition | | Non-empty target segmentation | | | | Empty target |
|---|---|---|---|---|---|---|
| Train | I | | HD 95% | CD | CD_{Heart} | False pos |
| strategy | minmax | Dice ↑ | (mm) ↓ | (mm) ↓ | (mm) ↓ | (vx) ↓ |
| nnU-net | self-configured | 0.629 ± | 8.315 ± | 1.478 ± | 0.929 ± | 2.2 ± |
| | | 0.158 | 10.688 | 1.910 | 0.188 | 6.4 |
| DiceCE | [−200, 1500] | 0.657 ± | 5.058 ± | 0.918 ± | 0.989 ± | 24.9 ± |
| | | 0.086 | 7.658 | 0.860 | 0.219 | 41.7 |
| GDL (ours) | [−200, 1500] | 0.657 ± | 5.083 ± | 0.834 ± | 0.976 ± | 22.8 ± |
| | | 0.079 | 7.277 | 0.758 | 0.206 | 31.0 |
| Gbd | [−200, 1500] | 0.632 ± | 4.973 ± | 0.906 ± | 0.979 ± | 2.9 ± |
| | | 0.113 | 5.074 | 0.644 | 0.209 | 4.1 |
| Gch | [−200, 1500] | 0.624 ± | 4.543 ± | 0.900 ± | 0.972 ± | 5.2 ± |
| | | 0.112 | 4.421 | 0.611 | 0.200 | 5.4 |

Same symbols as Table 1.

### 4.3 Segmentation Post-Processing Evaluation

For segmentation post-processing, we first evaluated its effects on the final segmentation's spatial accuracy and anatomical consistency (Table 2, FIGS. 6A-C).

Qualitative evaluations and the overall improvements in the mean surface accuracy metric (CD) indicate that our algorithm reasonably preserves the initial segmentation's spatial characteristics. Significant improvements in the calcification-to-heart distance (CDheart) indicates reduced spatial gaps and better anatomical consistency between calcification and its surrounding tissue.

The improved surface metrics HD and CD demonstrate our algorithm's effectiveness in filtering out easily avoidable false-positive calcification segments, such as those outside the aorta and those with tiny volumes. Interestingly, this filtering process reduced the performance gap between different segmentation models (Table. 1→Table. 2), which suggests that our postprocessing algorithm was effective at removing segmentation outliers for various models.

The worse Dice and better CDHeart demonstrates the tradeoff between volume overlap and anatomical consistency. This is expected, since the current strategy for removing the calcification-to-heart spatial gaps is to extend the calcification segments while keeping the heart mesh fixed. For the purposes of calcification meshing, the benefit of anatomical consistency far outweighs the slight reduction in volume overlap (FIG. 8). An interesting future direction is to jointly optimize both the heart mesh and the calcification segmentation to encourage anatomical consistency while minimizing this tradeoff.

### 4.4 Calcification Meshing Evaluation

The final desired output is a tetrahedral calcification mesh. To compare the robustness and quality of C-MAC vs. previous meshing approaches in generating this output, we calculated the following metrics: (1) the success rate of the final tetrahedralization step, (2) the spatial accuracy of the final mesh surface, and (3) the element quality of the final tetrahedra (Table. 3). The general strategy was to first construct a triangular surface mesh from a voxelgrid segmentation and then apply TetGen to tetrahedralize the inner volume.

For baselines, we first tried mesh boolean operations from a wide variety of existing meshing libraries. For 5 of the 8 libraries, the success rate for the final tetrahedralization was below 10% (Table. 3). For some, even the initial boolean operation failed completely. This is consistent with the notion that mesh boolean operations are generally unreliable, especially when coupled with complex downstream tasks such as constrained tetrahedralization.

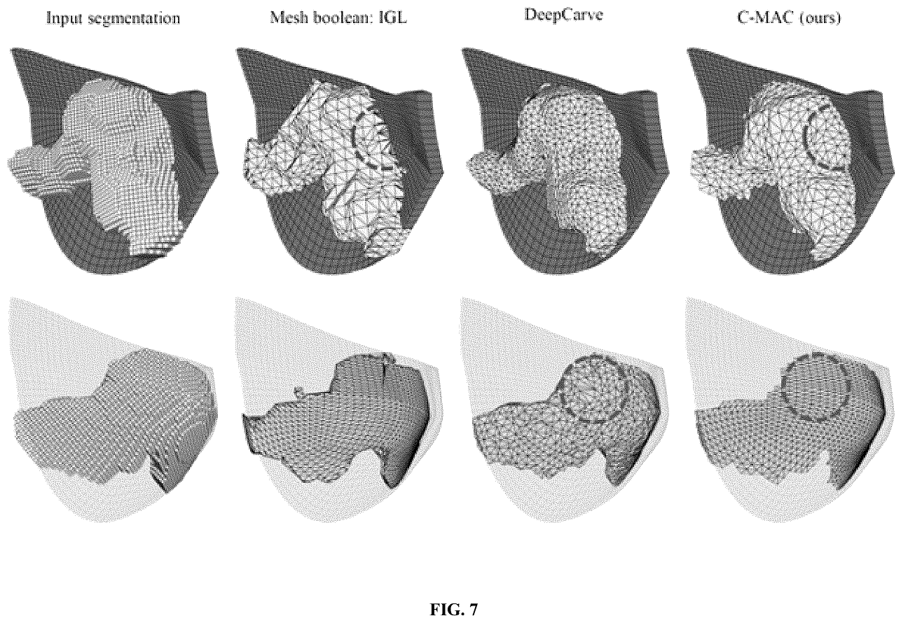

For mesh boolean operations with relatively high success rates, the issue still remained that the element qualities were heavily compromised along the mesh intersections. This is shown both qualitatively as thin triangular elements (FIG. 7), and quantitatively as low Jacobian determinants (Table. 3). Mesh boolean approaches had worse performance in essentially every category compared to our method.

As an additional baseline, we performed DeepCarve's node stitching operations as outlined in Pak et al. (2023). The only metric that performed well for this method was the Jacobian determinant, which makes sense given the generally smooth and uniform elements shown in FIG. 7. However, the method notably suffered from irregular node-stitching along the mesh intersections (FIG. 7), which is further confirmed by the low numbers of merged nodes (Table. 3). The irregular node-stitching also causes various other issues, such as the occasional undesired mesh intersections and failure of TetGen due to the weirdly shaped elements. Since it is difficult to automatically and robustly fix ill-formed meshes, our approach aims to generate good quality meshes from the beginning.

C-MAC is able to combine the best of both worlds by (1) establishing complete contact surface matching, similar to mesh boolean operations, and (2) providing great element quality along the non-contact surfaces, similar to DeepCarve's meshing approach. (FIG. 7). In addition, C-MAC ensures that any node residing on any of the heart surface's planes will be coincident with an existing heart mesh node, which prevents any potential issues with slight mesh intersections that can introduce cumbersome errors during simulations.

To demonstrate the importance of each of our meshing step, we compared the meshing performance between partial vs. full C-MAC (Table. 3). The two main processes are DMTetOpt and ConstrainedRemesh, so we performed a total of four experiments with and without either of those processes. As shown in Table. 3, the condition that includes both DMTetOpt and ConstrainedRemesh results in the best overall meshing performance, both in terms of spatial accuracy and element quality. Notably, DMTetOpt significantly improves all metrics, including the success rate of TetGen. ConstrainedRemesh is also visibly beneficial, but it can occasionally generate nonmanifold surfaces due to the way we separate contact and noncontact surfaces in our constrained clustering approach (Alg. 7). We are still able to reach 100% Tetgen success rate because we can easily store and select the optimally processed mesh at lower iterations of ConstrainedRemesh by simply running TetGen on all intermediate meshes.

TABLE 3
The effects of the calcification surface meshing
algorithms on the final tetrahedral mesh.

| | Success | | HD 95% | CD | Merged | |
|---|---|---|---|---|---|---|
| Mesh technique | (%)↑ | Dice ↑ | (mm) ↓ | (mm) ↓ | nodes ↑ | \| J \| ↑ |
| Bool: pymadcad | 0.0 | N/A | N/A | N/A | N/A | N/A |
| Bool: OpenSCAD | 0.0 | N/A | N/A | N/A | N/A | N/A |
| Bool: vtk | 0.0 | N/A | N/A | N/A | N/A | N/A |
| Bool: cgal | 3.7 | N/A | N/A | N/A | N/A | N/A |
| Bool: blender | 7.4 | 0.777 ± | 1.207 ± | 0.351 ± | 441 ± | 0.349 ± |
| | | 0.113 | 0.293 | 0.206 | 546 | 0.099 |
| Bool: cork | 70.0 | 0.794 ± | 1.399 ± | 0.384 ± | 3013 ± | 0.458 ± |
| | | 0.060 | 0.080 | 0.081 | 1742 | 0.043 |
| Bool: igl | 77.8 | 0.791 ± | 1.394 ± | 0.370 ± | 2718 ± | 0.449 ± |
| | | 0.066 | 0.090 | 0.080 | 1570 | 0.044 |
| Bool: corefinement | 81.5 | 0.793 ± | 1.395 ± | 0.370 ± | 2748 ± | 0.452 ± |
| | | 0.066 | 0.088 | 0.079 | 1539 | 0.043 |
| DeepCarve | 92.6 | 0.733 ± | 2.362 ± | 0.607 ± | 658 ± | 0.605 ± |
| | | 0.077 | 3.071 | 0.270 | 237 | 0.020 |
| DMTet (no opt) | 89.6 | 0.814 ± | 1.030 ± | 0.234 ± | 3582 ± | 0.547 ± |
| | | 0.044 | 0.075 | 0.017 | 1995 | 0.013 |
| DMTet (no opt) → | 99.3 | 0.819 ± | 1.041 ± | 0.237 ± | 3666 ± | 0.582 ± |
| ConstrainedRemesh | | 0.041 | 0.118 | 0.017 | 2024 | 0.008 |
| DMTetOpt | 100.0 | 0.827 ± | 1.019 ± | 0.225 ± | 3583 ± | 0.579 ± |
| | | 0.038 | 0.073 | 0.018 | 1995 | 0.008 |
| DMTetOpt → | 100.0 | 0.831 ± | 1.035 ± | 0.226 ± | 3576 ± | 0.588 ± |
| ConstrainedRemesh | | 0.037 | 0.108 | 0.018 | 1993 | 0.010 |
| (C-MAC) | | | | | | |

“Bool” refers to the mesh boolean difference operation between the original calcification surface and the aorta + aortic valve surface. All metrics were obtained using 10 repeated trials to account for the occasional failure of TetGen. We used the post-processed ground truth segmentation as the initial segmentation. Spatial accuracy metrics were measured against the initial segmentation, so we are strictly measuring the error induced by the meshing steps. All metrics are calculated using samples with non-empty targets only.

TABLE 4
Final tetrahedral calcification mesh metrics with
and without our post-processing algorithm.

| Experiment condition | | Non-empty target segmentation | | | | Empty target |
|---|---|---|---|---|---|---|
| Initial | Post- | Merged | | HD 95% | CD | False pos |
| segment | process | nodes ↑ | Dice ↑ | (mm) ↓ | (mm) ↓ | (vx) ↓ |
| Ground truth | No | 2620 ± | 0.760 ± | 1.042 ± | 0.321 ± | N/A |
| | | 1657 | 0.039 | 0.100 | 0.042 | |
| Ground truth | Yes | 3298 ± | 0.671 ± | 1.806 ± | 0.528 ± | N/A |
| | | 1907 | 0.048 | 1.294 | 0.139 | |
| GDL (ours) | No | 1900 ± | 0.673 ± | 3.774 ± | 0.794 ± | 11.3 ± |
| | | 817 | 0.084 | 4.439 | 0.924 | 15.2 |
| GDL (ours) | Yes | 2686 ± | 0.621 ± | 5.281 ± | 0.913 ± | 18.1 ± |
| | | 1101 | 0.081 | 7.178 | 0.737 | 23.9 |

Similar to Table 3, we used 10 repeated trials to obtain these metrics. Spatial accuracy metrics were measured against the ground-truth segmentation. Merged nodes indicate the number of the final calcification mesh nodes within 1e−3 mm from the heart mesh nodes.

As our final geometrical evaluation, we measured the effects of our segmentation post-processing algorithm on the final CMAC meshes, using both the ground truth and the deep learning model prediction as our initial segmentation. The observations are very similar to the previous segmentation post-processing evaluations. The anatomical consistency, represented by the number of merged nodes between the final calcification mesh and the heart surfaces, was drastically improved for both the ground-truth and DL-predicted initial segmentation (Table 4, FIG. 8). The overall spatial accuracy was reduced due to the nature of our algorithm, which fixes the heart geometry and only alters the calcification segmentation to encourage anatomical consistency (Table 4). The slight overestimation caused by the segmentation post-processing algorithm is also illustrated in FIG. 8.

### 4.5 Run-Time

The approximate run-time to generate the entire calcified heart geometry (i.e. DeepCarve+C-MAC) is ˜1 minute per 3D image. The three most time-consuming tasks are (1) closing operations for segmentation post-processing (˜10 seconds), (2) TetGen for background mesh generation (˜15 seconds), and (3) DMTet optimization (˜20 seconds). Most other individual processes typically take <1 second.

### 4.6 Solid Mechanics Simulations

We performed two sets of solid mechanics simulations to assess different aspects of our final meshes. First is valve opening simulations, which demonstrate our method's unique ability to set up large-scale simulations. Second is TAVR stent deployment simulations, which demonstrate our meshes' robustness to complex simulation environments.

For valve opening, a uniform pressure of 8 mmHg was applied to the ventricular surface of the leaflets using Abaqus (3DS, [symbol] , Paris, France). The pressure was increased compared to previous work (Martin and Sun, 2015) to represent elevated aortic valve pressure gradient in mild aortic stenosis.

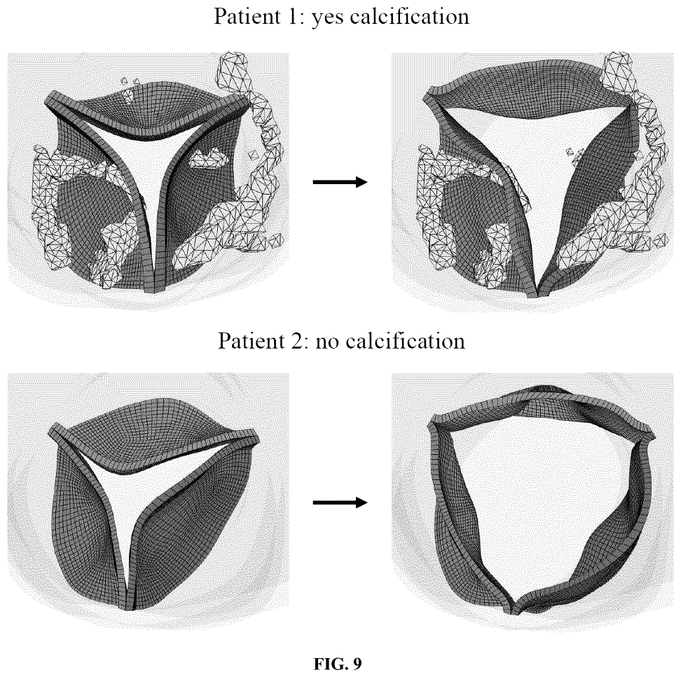

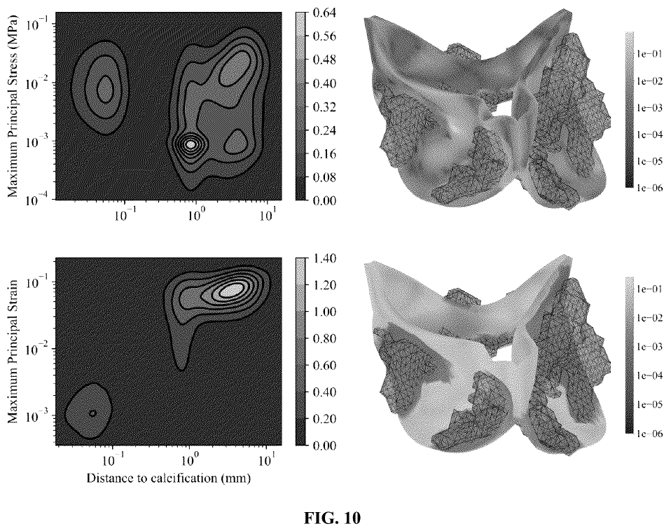

From these simulations, we extracted two main pieces of information. First is the effect of the calcification on the leaflet movement. As shown in FIG. 9, the leaflet movement is clearly restricted near the calcified regions. This can be attributed to the high stiffness of the calcification and the aortic wall, which incur both intrinsic resistance from the leaflet elements and external push-back from the physical connection to the aortic wall. Second set of information was the stress/strain values. As shown in FIG. 10, stress/strain values are significantly influenced by the location of the calcification. The simulations were performed using completely auto-generated meshes from Deep-Carve and C-MAC, and the stress/strain results correspond well to the location of the ground-truth calcification.

Valve opening simulations were performed across all 35 patients with fully automated simulation setups. This was possible because we maintained the mesh correspondence of heart meshes, and all boundary and loading conditions were defined with respect to the mesh elements. This allowed us to both qualitatively confirm that our results and observations were consistent across a large number of samples, and also quantify our observations using the test-set population results. In this study, we performed Gaussian kernel density estimation (KDE) (Scott, 2015; Virtanen et al., 2020) on the aggregated data points of all test-set simulations to estimate the joint distribution of the leaflet nodes' stress/strain values vs. their distances to the nearest calcification. The KDE plots in FIG. 10 both confirmed our qualitative observations that nodes closer to calcification have low strain, and revealed new insights that the leaflet stress has a complex distribution at 1-10 millimeters away from the calcification.

For TAVR stent deployment, we modeled a 26 mm, self-expandable, first-generation Medtronic CoreValve device (Medtronic, Minneapolis, MN, USA) following previous works (Mao et al., 2018; Caballero et al., 2020). We selected 10 patient-specific anatomies that are suitable for this device size according to the manufacturer's recommendation of CoreValve system (Medtronic LLC, 2017). Briefly, the crimped TAVR stent with an exterior diamater of 6 mm (Martin and Sun, 2015) was aligned coaxially within the aortic root and centered into the aortic annulus, following the manufacturer's recommendations (Medtronic LLC, 2017). The stent was deployed inside the aortic root by axially moving the cylindrical sheath towards the ascending aorta.

Using the original calcification meshing algorithm from DeepCarve, 4 out of the 10 simulations failed due to calcification element distortions. Using C-MAC, all 10 simulations successfully ran to completion due to the higher quality calcification elements. The simulation results concur with previous observations that the calcified regions are associated with higher levels of stress. This further confirms the high robustness of our meshing algorithm and the benefit it provides for complex downstream tasks.

### 5. DISCUSSION

Our extensive evaluations suggest that C-MAC is a robust fully automated solution for incorporating aortic calcification mesh into an existing heart mesh. (1) Our deep learning segmentation model provides a good initial calcification segmentation. (2) Our segmentation post-processing algorithm enhances the calcification-to-heart anatomical consistency while slightly compromising the segmentation volume accuracy. (3) Our meshing algorithm accurately converts the input voxelgrid segmentation into surface meshes with great element quality along both contact and non-contact surfaces. The meshing algorithm also preserves the original heart mesh topology.

Unlike the original DMTet paper (Shen et al., 2021), we did not train a separate neural network to expedite the DMTet optimization process. Instead, we opted for a per-image optimization approach. The main reason for this choice was the long processing time of TetGen for the background mesh generation (Section 4.5). Unlike Shen et al. (2021) where the background mesh was a fixed lattice of regularly spaced tetrahedra, our meshing workflow requires a background mesh that contains the patient-specific heart geometry in the inner volume (FIG. 4). Since the element density and configuration can change drastically based on the leaflet conformation, we could not reliably construct the background mesh without repeating TetGen for each scan separately. This poses a significant restriction on our training scheme since repeating TetGen for every augmented training sample would significantly increase the training time (from ˜1 second to ˜15 seconds per sample) or storage (˜20 MB per background mesh for hundreds of thousands of samples).

Furthermore, even if we were able to successfully train a deep learning model for the DMTet optimization process, it would only reduce the total run-time from ˜1 minute to ˜40 seconds because the other major time-consuming processes have nothing to do with DMTet optimization (Section 4.5). Therefore, we decided to simply use the proposed per-image optimization approach. This approach has an additional minor benefit that we can slightly randomize the output with different initial 6 if we are unsatisfied with our output.

Our current approach for PostProcessCa2Seg has a tradeoff between calcification segmentation accuracy and anatomical consistency because we fix DeepCarve's heart structures while extending the calcification segments to remove the spatial gaps. We aimed to achieve the best possible solution for this approach using an adaptive closing kernel, but the inherent reduction in volume overlap is most likely unavoidable. An interesting future direction would be to jointly optimize the heart structures and the calcification segments to deform the heart structures towards the predicted calcification, instead of simply extending the calcification. This approach is technically more challenging because it requires simultaneous optimization of multiple metrics, such as the calcification accuracy, heart surface accuracy, heart mesh element quality, and also requires a robust formulation for the selective attraction between only the contact surfaces of the heart and calcification meshes.

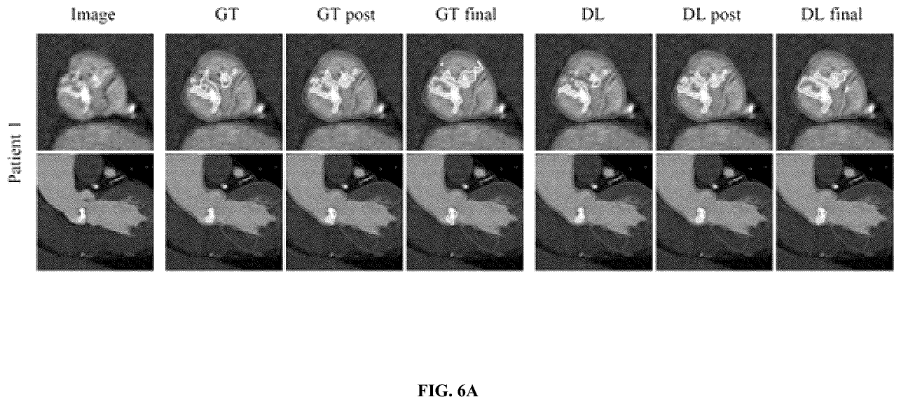

Another important consideration for C-MAC is the usability of the final meshes. Since we optimize for good element quality using DMTetOpt, the key remaining consideration is the manifoldness of the output. As mentioned in Section 2, one of the main strengths of marching tetrahedra is its ability to generate topologically consistent outputs due to its unambiguous tessellation of the tetrahedral lattice. The same principle applies to the raw non-optimized DMTet output with our custom background mesh and calcification segmentation, which means the output should be manifold surfaces without any modifications. The main issue with the non-optimized DMTet output is that TetGen can occasionally fail due to the highly irregular triangular elements from the raw node-sampled SDF (FIG. 5, DMTet initial).

We proposed DMTetOpt to maintain the original manifoldness of DMTet while improving the element quality. The element quality improvements are straightforwardly obtained using [symbol] _{DMTet} (Eq. 8), which optimizes for various desired triangle properties. For the manifoldness, we first argue that ModifyEdgeCases does not alter the manifoldness of the original DMTet output. From Eq. 7: (1) For any tetrahedron with an SDF sign-change across the fake node, the output surface will always be a heart surface element. DeepCarve's heart meshes all have manifold surfaces by design, so the DMTet output at contact surfaces will also be manifold. (2) For any tetrahedron with an SDF sign-change across a boundary node, the output surface will always only expand its borders along the boundary surface, and the amount of expansion will at worst cause coincident faces. This means no self-intersections will be introduced by our modification, so the DMTet output will be manifold.

For DMTetOpt's δ optimization, ΔSDF is constrained to only change each nodal SDF's magnitude while keeping its original sign, which prevents any topological changes. The only remaining potential concern is ΔVbg. Given that [symbol] _{DMTet} is only calculated for tetrahedra with nodal SDF sign-changes, the ΔV_{bg} optimization will also only be applied to those elements' nodes. In this case, a potential way for ΔV_{bg} to cause non-manifoldness is to cause self-intersections by heavily displacing the background mesh nodes near the segmentation isosurface. This is strongly discouraged by many of our design choices, such as (1) [symbol] _{DMTet}'S surface smoothness constraints and ΔV_{bg} regularization and (2) our strategy of updating the nodal SDF at the new node coordinate for each DMTetOpt iteration. Empirically, DMTetOpt consistently generates high quality manifold meshes, leading to a TetGen success rate of 100%.

We further processed DMTetOpt's output mesh with ConstrainedRemesh for better element quality, but the remeshing step could occasionally introduce non-manifold surfaces due to (1) the nature of ACVD's vertex clustering algorithm and (2) our strategy of separating the contact and noncontact surfaces for remeshing. Nonetheless, we can still empirically obtain a TetGen success rate of 100% for DMTetOpt→ConstrainedRemesh by simply running the algorithm nᵣₑₘₑₛₕ times, trying TetGen with the output at each iteration, and selecting the latest mesh with a successful TetGen output. This is possible because both ConstrainedRemesh and TetGen take a fraction of a second to perform, and the outputs are also not very memory intensive.

Clinical validation of solid mechanics simulations is very challenging because post-TAVR CT's are not routinely collected and the ground-truth stress/strain values for in vivo anatomical structures are nearly impossible to obtain. The solid mechanics simulations in this work instead illustrate two other important points: (1) our generated meshes can handle complex solid interactions such as TAVR stent deployments and (2) the high-quality mesh output with mesh correspondence allows for large-batch simulations, even with the addition of calcification. The accuracy of our simulations will be illustrated through flow-based simulations in future studies using the more routinely collected Doppler echo data.

### 6. CONCLUSION

In this work, we propose an automated end-to-end image-to-mesh solution for incorporating calcification into an existing heart geometry. Our two-part solution includes (1) a segmentation algorithm that encourages anatomical consistency between the predicted calcification and heart mesh and (2) a meshing algorithm that establishes complete contact surface matching between the two geometries while maintaining the original heart mesh topology. Our technique allows for large-batch simulations due to the preservation of inter-patient mesh correspondence of the heart geometry. We demonstrated the viability of such downstream tasks using solid mechanics simulations.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An automated method of incorporating sporadic objects (SO) onto an existing object (EO) mesh, the method comprising:
providing an EO mesh;
providing an SO voxelgrid segmentation; and
applying an SO meshing algorithm, the SO meshing algorithm generating an SO surface mesh from the combination of the EO mesh and the SO voxelgrid segmentation;
wherein the SO mesh has complete mesh correspondence with the EO mesh along contact surfaces;
wherein the SO mesh is free of mesh degeneracy; and
wherein the EO mesh topology is unchanged.

2. The method of claim 1, wherein the EO mesh includes a surface mesh, a volumetric mesh, or a combination thereof.

3. The method of claim 1, further comprising post-processing the SO voxelgrid segmentation, the post-processing removing undesired spatial gaps between the SO voxelgrid segmentation and the EO mesh.

4. The method of claim 3, wherein the post-processing step includes:
converting the EO mesh into EO voxelgrid segmentation;
dilating the SO voxelgrid segmentation;
keeping only segments of the EO voxelgrid segmentation that intersect with segments of the dilated SO voxelgrid segmentation;
combining the EO and SO voxelgrid segmentations;
morphologically closing the combined voxelgrid segmentations; and
filtering the combined voxelgrid segmentations by subtracting the EO segments and performing volume-based island removals.

5. The method of claim 4, wherein the SO voxelgrid segmentation comprise calcification segments.

6. The method of claim 5, further comprising, prior to the converting step, splitting the calcification segments into disjoint groups to prevent over-dilation (i.e. overly connected components).

7. The method of claim 4, wherein the dilation and morphological closing operations are performed using combinations of isotropic and/or anisotropic filters.

8. The method of claim 7, wherein the anisotropy is defined as the major principal axis pointing in the same direction as the nearest EO mesh node's surface normal.

9. The method of claim 1, wherein the SO meshing algorithm comprises:
generating a background tetrahedral mesh;
converting the SO segmentation to a nodal signed distance function (SDF);
generating an isosurface triangular mesh from the background mesh and nodal SDF using marching tetrahedra; and
simultaneously optimizing the nodal SDF and the background mesh, forming an optimized SO surface mesh.

10. The method of claim 9, wherein generating the background mesh includes:
extracting the EO surface;
processing the surface for constrained tetrahedral meshing;
performing constrained tetrahedral meshing; and
adding a fake node to all background boundary surface elements to form fake tetrahedral elements.

11. The method of claim 10, wherein generating the background mesh further includes:
after extracting the EO surface, generating an offset surface including all areas within a pre-defined voxel spacing away from the surface of the region of interest;
combining the extracted surface and the offset surface, forming merged surfaces for tetrahedral meshing;
performing the constrained tetrahedral meshing with the merged surfaces; and
before adding the fake node, removing elements inside the original EO surface.

12. The method of claim 9, wherein converting the SO segmentation to a nodal signed distance functions (SDF) comprises:
converting the SO segmentation to a voxelgrid SDF via linear mapping or voxelwise distance calculation from the SO isosurface; and
interpolating the voxelgrid SDF at each background mesh node, forming nodal SDF.

13. The method of claim 9, wherein optimizing the nodal SDF and the background mesh comprises adjusting the nodal SDF and the mesh vertex positions to maximize surface mesh quality and minimize deviation from the SO voxelgrid segmentation.

14. The method of claim 13, further comprising setting the nodal SDF values to prescribed values when the nodes are from the original EO mesh nodes or the fake node.

15. The method of claim 9, further comprising remeshing non-contact surfaces after generating the isosurface triangular mesh of the SO surface, the remeshing of the non-contact surfaces reducing complexity of the mesh.

16. The method of claim 15, wherein the remeshing comprises constrained surface remeshing.

17. The method of claim 16, wherein the constrained surface remeshing comprises:
splitting the optimized SO surface mesh into two disjoint sets of contact and non-contact elements, each set of elements representing a separate surface mesh;
determining that an element is a contact element when all three nodes of a triangular element are coincident with the original mesh nodes;
performing an initial vertex-based clustering step on the non-contact surface mesh;
reassigning the clusters at overlapping nodes between the contact and non-contact surface meshes;
performing a standard vertex clustering-based triangulation, providing a remeshed non-contact surface mesh; and
merging the contact surface mesh and the remeshed non-contact surface mesh to obtain a final output surface mesh.

18. The method of claim 17, further comprising repeating the remeshing to achieve a desired element size and density.

19. The method of claim 1, further comprising:
after applying the SO meshing algorithm, applying a tetrahedralization algorithm to the SO surface mesh, the tetrahedralization algorithm converting the SO surface mesh to a tetrahedral SO volumetric mesh; and
replacing the SO surface mesh with the SO volumetric mesh in the subsequent merging step.

20. The method of claim 1, further comprising:
merging the SO surface/volumetric mesh with the EO mesh via coincident nodes.

21. An apparatus for automated calcification meshing with anatomical consistency, the apparatus comprising:
a processor;
a memory unit; and
a communication interface;
wherein the processor is connected to the memory unit and the communication interface; and
wherein the processor and memory are configured to implement the method of claim 1.

22. A non-transitory computer readable storage medium storing computer-executable instructions for performing the method of claim 1.
